(19) United States
(12) Patent Application Publication
(10) Pub. No.: US 2006/0263441 A1
(43) Pub. Date: Nov. 23, 2006

(54) Free radical solution water

(76) Inventors: Kenji Fukui, Tokyo (JP); Kazunari Naya, Tokyo (JP)

Correspondence Address: GREENBERG TRAURIG LLP, 2450 COLORADO AVENUE, SUITE 400E, SANTA MONICA, CA, 90404, US

(21) Appl. No.: 10/547,722

(22) PCT Filed: Mar. 4, 2003
(86) PCT No.: PCT/US03/06600

§ 371 Date: May 30, 2006

(51) Int. Cl. A61K 33/00

(52) U.S. Cl. 424/600

(57) ABSTRACT

Free Radical Solution functional (electrolyzed) water with high oxidation reduction potential of 900 mV, and with a stable Hydrogen Ion Concentration (HIC) level of 6-8 pH is provided that cleans, deodorizes, and sterilizes without any chemicals. The FRS water is pollution free, non-chemical and water based solution. The original and the resulting end material (or component) in the process of producing FRS water is simply water (H₂O). Using transformation of water molecules by pretreatment and electrolysis processes, water is transformed to Free Radical Solution water wherein the free radicals in the FRS water solution add the very unique (physical) characteristics and functions that makes this water different from regular water (physically). The transformation is not chemical, but a physical change of atoms or molecules in water, i.e. the H₂O molecules of water are transformed into different types of free radicals. The transformations are random, continuous, and repeat for at least two hours after production.

### TECHNICAL FIELD

The present invention relates to water, and more particularly to Free Radical Solution (FRS) functional (electrolyzed) water.

### BACKGROUND ART

There is a growing interest in special kinds of treated water with physical properties that differ from those of ordinary water. Treaded water is water that has been processed through a variety of methods such as electrolytic ion separation or electrolysis to produce what are known as functional waters with unique physical characteristics. The processes that produce functional water systems restructure regular water into different types of functional (or utility) waters, including for example waters with alkaline concentrations or acidic concentrations. These waters may be used for sterilization in settings such as hospitals, medical clinics, or other industries such as food processing, or households, similar to chemical-based sterilizing solutions. Ultra Acidic Water is a typical example of a functional water used as an oxidizing agent (a bleaching product) for sterilization. The highly acidic nature of this water (about 2-3 pH) limits its use, and may be harmful for the environment. In fact, the Ultra Acidic Water and many other prior art functional waters (such as alkaline waters) may require a neutralization solution for their proper and safe disposal. In addition, like all other prior art functional waters, the Ultra Acidic water is produced by the addition of chemical additives during various stages of water treatment, including electrolysis. Water is a weak electrolyte because it conducts very little amount of electric current. In order to electrolyze water efficiently, a small amount of additive such as salt or sulfate is added, making the resulting solution an electrolyte. The electrolyte is than placed in a tank (known as electrolytic tank) divided into two by a diaphragm (or a membrane). When electric current is passed through water, between the cathode on one side of the diaphragm (or membrane) and the anode electrode on the other, H³⁰ ions are deposited at cathode and OH³¹ ions are deposited at anode sections of diaphragm (or membrane), producing electrolyzed water.

In addition to chemical additives to facilitate the electrolysis process, the resulting prior art electrolyzed waters generated are either acidic or alkaline with very high effective chlorine levels, further limiting their application.

### DISCLOSURE OF INVENTION

The present invention seeks to provide electrolyzed functional water that is pollution free and can be used as cleaning, deodorizing, and sterilizing solution.

The present invention further seeks to provide electrolyzed functional water without the addition of any additives (chemical or otherwise).

In addition, the present invention seeks to provide electrolyzed functional water with Oxidation-Reduction Potential level of 900 mV to 1200 mV, and with a stable Hydrogen Ion Concentration level of 6-8 pH.

In keeping with the principles of the present invention, unique functional (electrolyzed) water is presented that overcomes the short falls of the prior art functional waters. The Free Radical Solution (FRS) water of the present invention is functional (electrolyzed) water with no additives (chemical or otherwise). The FRS water is non-chemical and water based solution that has very strong cleansing, deodorizing and sterilizing capabilities. The original material used in the process of producing FRS water, and the FRS water itself, is simply water (H₂O). Its difference with regular water has to do with its physical characteristics, making it distinctly different from water's more common well-known chemical features. Using transformation of water molecules in an environment created by pretreatment and electrolysis processes, water is transformed to Free Radical Solution water wherein the free radicals in the FRS water solution add the very unique characteristics and functions that makes this water different from regular water (physically). The transformation is not chemical, but a physical change of atoms or molecules in water, i.e. the H₂O molecules of water are transformed into different types of free radicals. The transformations are random, continuous, and repeat.

In general, after processing of water, the water molecules are transformed into Free Radical mono-molecules, with the resulting FRS water having a stable Hydrogen Ion Concentration (HIC) level of 6 to 8 pH, with Oxidation Reduction Potential (ORP) of about 900 mV to 1200 mV. The FRS water service life time when exposed to air is at least two hours after production, where it gradually returns to regular water thereafter. The high ORP levels in FRS water allow it be used for generating electricity.

These and other objects, features, aspects, and advantages of the invention will be apparent to those skilled in the art from the following detailed description of preferred non-limiting embodiments, taken together with the drawings and the claims that follow.

### BRIEF DESCRIPTION OF DRAWINGS

It is to be understood that the drawings are to be used for the purposes of illustration only and not as a definition of the limits of the invention.

Referring to the drawings in which like reference numbers present corresponding parts throughout:

FIG. 1 is a summary table showing the properties and the corresponding values for Free Radical Solution water, in accordance with the present invention;

FIG. 2 is a flow chart illustrating the process of producing the FRS water, in accordance with the present invention;

FIG. 3 is a graphical representation of the result of a sample spectrum transmittance test for FRS water and purified water by an optical spectrum analyzer, in accordance with the present invention; FIG. 3A is a graphical representation of the result of a sample spectrum transmittance test for purified water; FIG. 3B is a graphical representation of the result of a sample spectrum transmittance test for FRS water.

FIG. 4 is a listing of Free Radicals and their transformation patterns in FRS water, in accordance with the present invention;

FIG. 5 is a graphical representation of a sample Electron Spin Resonance (ESR) test result for capture of *OH radicals in FRS water, in accordance with the present invention;

FIG. 6 is a graphical representation for the Oxidation Reduction Potential change pattern for FRS water, in accordance with the present invention;

FIG. 7 is a graphical representation, illustrating power consumption during electrolysis for the production of FRS water, in accordance with the present invention;

FIG. 8 is a comparison table illustrating the differences between the FRS water of the present invention and other existing prior art electrolysis water.

### BEST MODE FOR CARRYING OUT THE INVENTION

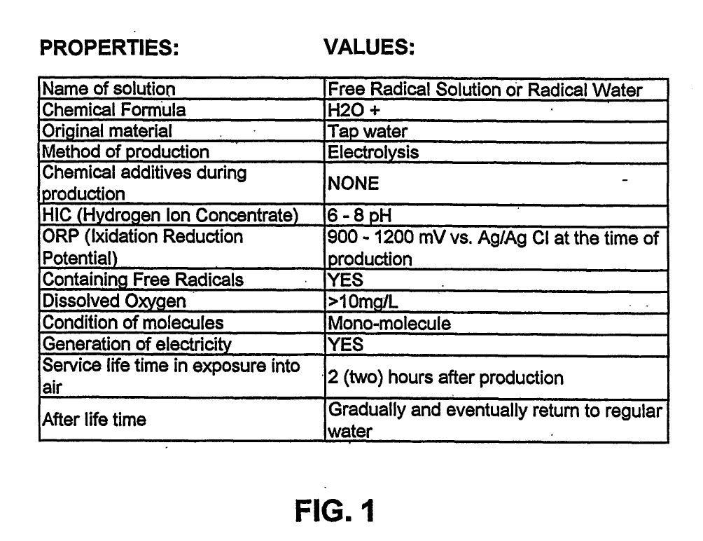

FIG. 1 is a summary illustration of the various properties and the corresponding values of Free Radical Solution (FRS) water. Chemically, FRS water comprises of H₂O+ molecules, and contains Free Radicals therein. Its Hydrogen Ion Concentration (HIC) level is stabilized at 6-8 pH, with Oxidation-Reduction Potential (ORP) of 900 mV to 1200 mV. In general, both ORP and HIC are used as a means to measure chemical characteristics of solutions, including water. The unit of measurement for HIC is “pH”, which stands for “potential for Hydrogen” and represents the negative logarithm of the Hydrogen Ion Concentration, and is a value that indicates whether a substance is either acidic or alkaline according to the following scale:
pH 1-3 Ultra Acidic
pH 4-5 Acidic
pH 6-8 Neutral
pH 9-11 Alkaline
pH 12 or more Ultra Alkaline
   The stated Hydrogen Ion Concentration (HIC) level of 6-8 pH is important in that it makes FRS water a safe neutral solution that does not require further processing for proper disposal. The range of 6-8 pH is also very important because it is not harmful for, use, and hence has a wider application. The Oxidation-Reduction Potential (ORP) defines the capability of a substance to either release or gain free electrons (the substance that gains electrons is termed the oxidizing agent). The unit of measurement for ORP is in general expressed in milli-volts (mV). Regular tap water for example has about 200 mV to 600 mV of ORP. The higher the ORP level of a substance, the stronger its sterilizing capability. The Dissolved Oxygen (DO) level of FRS water is greater than 10 mg/L, with effective chlorine level of 0˜1 mg/L (or PPM). Effective chlorine is a measure of the volume of chlorine in a solution. This low level enables the solution to be virtually free of chlorine, a chemical found in most water base products. The service lifetime when exposed to air for FRS water is at least two- (2) hours after production, and gradually returns to regular water thereafter. Due in part to its high Oxidation-Reduction Potential, the FRS water may also be used for generation of electricity.

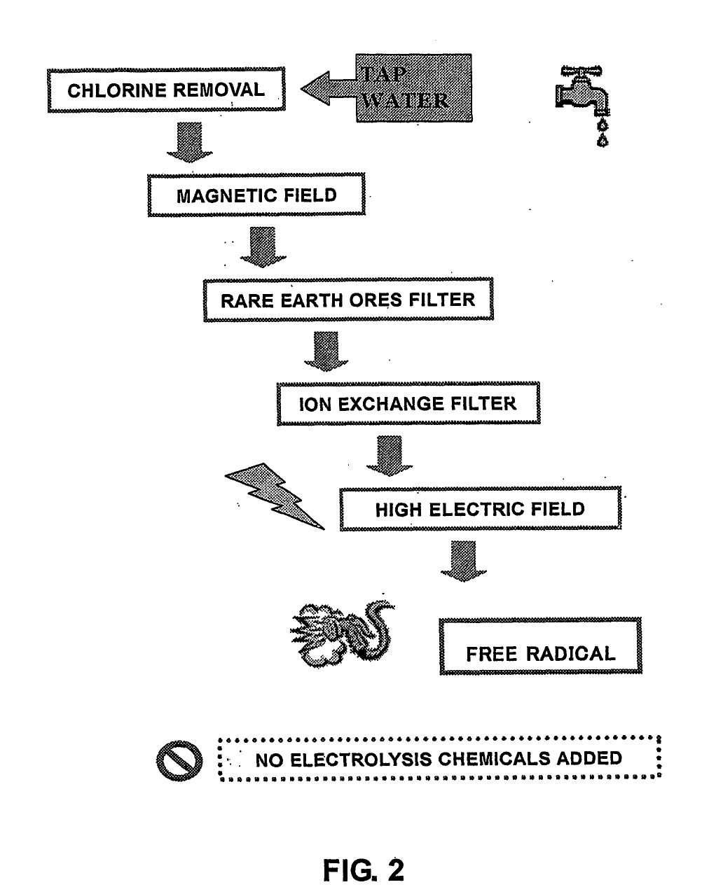

FIG. 2 is a flow chart illustrating the typical production process of FRS water. Prior to electrolysis process, regular water 2 is pre-treated through processes 4 to 10 to eliminate most impurities and prepare the water for electrolysis process 12 to produce Free Radical solution 14. As the name indicates, the Chlorine Removal Process 4 remove chlorine found in regular water. The removal of chlorine in the first step of pre-treatment of water 2 improves the overall electrolysis process in that no harmful chlorine gases will be generated when water is electrolyzed. The average volume of chlorine in regular water will depend on the water municipality and the jurisdictional regulations thereof. In general, regular water has around 20-30 mg/L of chlorine. After the Chlorine Removal Process 4, it will posses about 0-1 mg/L of chlorine.

The Magnetic Field Process 6 is a well-known method for separating H₂O water molecules, which are usually tied closely together in clusters, into single individual molecules of H₂O (known as mono-molecules). H₂O water molecules in water tend to cluster together, and with this process, H₂O water molecule clusters are separated into individual H₂O water molecules. This separation of H₂O water molecules improves the efficiency of the electrolysis process because H₂O water molecule clusters require a greater input of energy to be transformed into Free Radicals. This is because H₂O water molecules clusters are less energetic. The individual H₂O water mono-molecules on the other hand, tend to move randomly and more energetically in comparison with H₂O water molecule clusters. The more energetic and random a molecule moves, the easier its transformation will be to a Free Radical. In addition, this process also aids the H₂O mono-molecules in increasing the strength of their electrons, as magnets are widely used for enhancing electrons in various applications such as microwave, magnet generation, etc.

The Rare Earth Ores Ceramic Filter Process 8, finally transforms any remaining H₂O water molecule clusters remaining from the Magnetic Field Process 6, into single individual H₂O mono-molecules. The rare earth ores are comprised of such elements as Nd, Ce, La, Zr, Y, U, Tr, Pd, Fe, Gd, Ti, Ca, K, P, Si, and Al. The natural radiation from these materials further help separate H₂O water molecule clusters into individual H₂O water mono-molecules, further facilitating efficient electrolysis of water to produce Free Radical Solution water.

The Ion Exchange Filter Process 10 is used to remove “hardness” from water caused by materials such as calcium and Magnesium. These elements deteriorate the efficiency of the electrolysis process because they tend to combine with other molecules and create unwanted chemical reactions, hindering the electrolysis of water. This process can also be used to remove unwanted ions from polluted water streams. If regular water or pre-treated water is not available in service places such as remote disaster area, a supplemental filtration system is required before pretreatment processes 4 to 10.

The pretreated water is electrolyzed using High Electric Field Electrolysis Process 12, with no chemicals added. The electrolysis of water produces the Free Radical Solution 14 of the present invention. The pretreatment processes 4 to 10 remove most chemical composite materials other than H₂O such as for example, chlorine, magnesium, calcium, etc. In producing FRS water, all that is required is H₂O. The pre-treated water before electrolysis 12 is therefore soft water, almost equal to purified water. Through pre-treatment processes, regular water is purified and most of the impurities are eliminated to improve the efficiency of electrolysis. The electrolysis of water enhances its ORP to more than 900 mV while stabilizing its HIC within the range 6-8 pH.

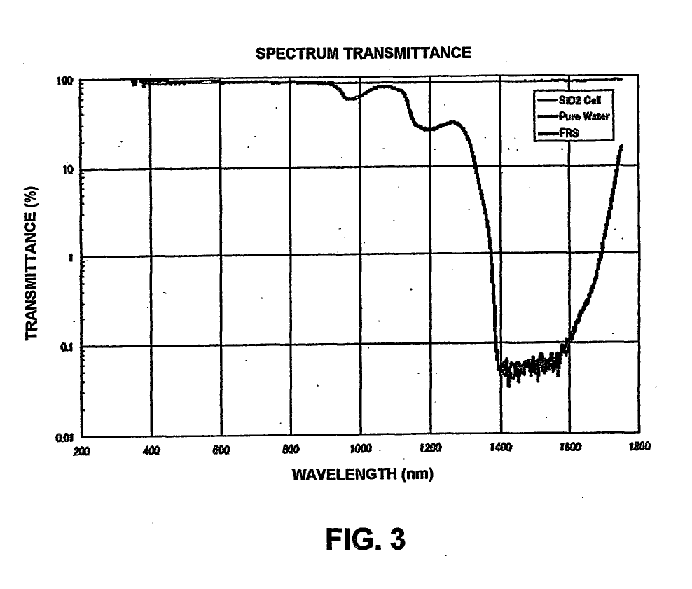

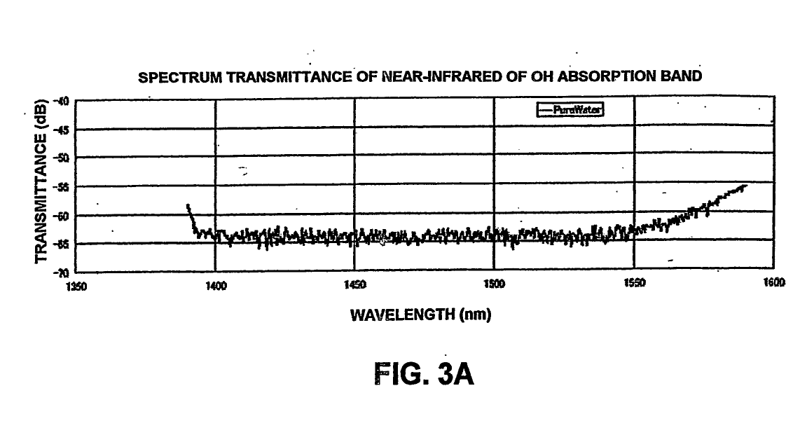

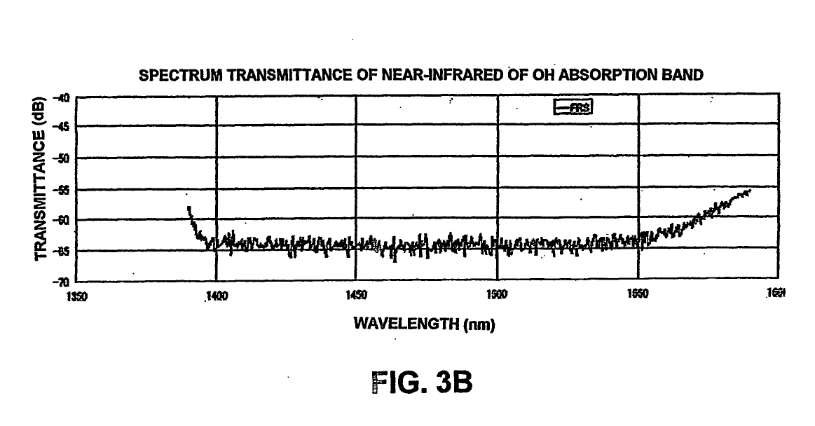

FIG. 3 is a sample of spectrum transmittance test result for FRS water and purified water. An optical spectrum analyzer measures the amount of spectrum absorbed (penetrated) and transmitted by a test material. Spectrum absorption and transmittance pattern for tested materials depend on the chemical composites contained therein. In this case, tested materials were FRS water and pure water. The result indicates that FRS water is almost identical to purified water because the transmittance patterns for both waters are almost identical as shown in FIG. 3A (purified water) and FIG. 3B (FRS water). The data proves that FRS water does not contain any chemical additives, and in fact, chemically is H₂O.

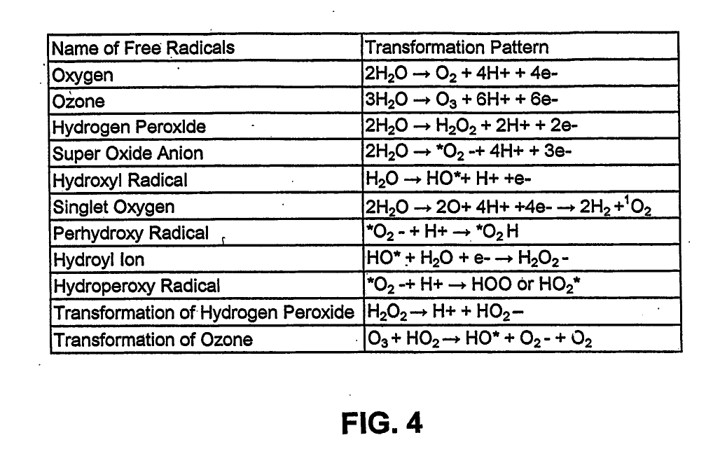

FIG. 4 is a listing of Free Radicals and their transformation patterns in FRS water. Transformation patterns illustrated in FIG. 4 occur repeatedly at random both during and after the electrolysis process. Each Free Radical is a result of the transformation of H₂O water molecules in FRS water, illustrating the difference between FRS water and regular water (where no H₂O molecule transformation occurs). Below is the same listing of transformation patterns and the resulting generated Free Radical:

| Transformation Pattern: | Free Radical Generated: |
|---|---|
| 2H₂O → O₂ + 4H⁺ + 4e⁻ | Oxygen |
| 3H₂O → O₃ + 6H⁺ + 6e⁻ | Ozone |
| 2H₂O → H₂O₂ + 2H⁺ + 2e⁻ | Hydrogen Peroxide |
| 2H₂O → *O₂_{^{−}} + 4H⁺ + 3e⁻ | Super Oxide Anion |
| H₂O → HO* + H⁺ + e⁻ | Hydroxyl Radical |
| 2H₂O → 2O + 4H⁺ + 4e⁻ → 2H₂ + ¹O₂ | Singlet Oxygen |
| *O₂⁻ + H⁺ → *O₂H | Perhydroxy Radical |
| HO* + H₂O + e⁻ → H₃O₂_{^{−}} | Hydroxyl Ion |
| *O₂⁻ + H⁺ → HOO or HO₂* | Hydroperoxy Radical |

The generation of Free Radicals is random and is not necessarily in the sequence listed both above and in FIG. 4. The Free Radicals are transformed from one to another instantly and frequently in a random manner. Super oxide anion and hydroxyl radical float as free radicals in FRS water and are eventually stabilized by transforming to ozone or hydrogen peroxide. Furthermore, hydrogen peroxide and ozone also continue their transformations after electrolysis in the following manner:

| | |
|---|---|
| H₂O₂ → H⁺ + HO₂_{^{−}} | (Transformation of hydrogen |
| | peroxide) |
| O₃ + HO₂ → HO* + O₂⁻ + O₂ | (Transformation of Ozone) |

Free Radicals generated are very unstable and tend to combine with other molecules or atoms surrounding them to stabilize. This phenomenon is used to sterilize various infective bacterial diseases such as for example, pathogenic bacilli. Application of FRS water to an infected area causes the Free Radicals in FRS water to combine with bacteria and other molecules, sterilizing the infected region. When water is electrolyzed, oxygen is forcefully removed from H₂O water molecules, and is transformed into one of unstable Free Radical atoms that tends to combine with other atoms or molecules surrounding it. The combinations of Free Radicals such as oxygen with other molecules, such as pathogenic bacilli oxidize and sterilize the bacteria. It is important to note that all the transformation patterns of Free Radicals shown in FIG. 4 are random, continuous and repeat at almost the same level for at least two- (2) hours after electrolysis.

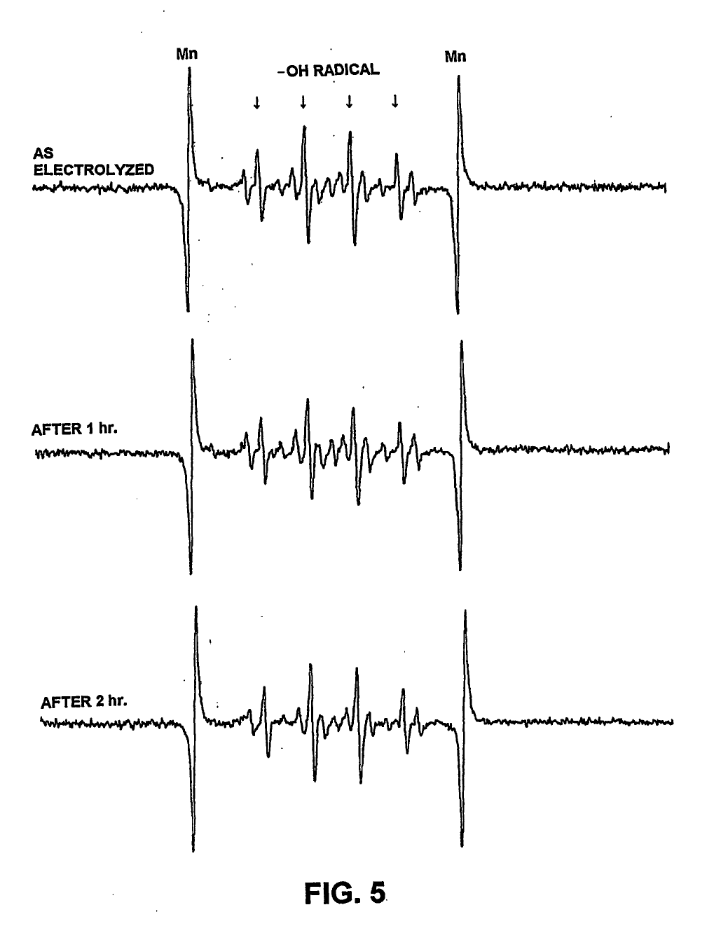

Various tests have shown that FRS water maintains a high level of ORP at least two hours after production. FIG. 5 is a sample Electron Spin Resonance (ESR) test result for capturing *OH radicals in FRS water detected immediately after production, one- (1) hour after production, and two- (2) hours after production. ESR spectrum patterns of all three are almost identical and show the capture of *OH radicals, which indicates that FRS water maintains the same level and transformations of Free Radicals consistently for at least two- (2) hours after production. The high ORP level of more than 900 mV generated in FRS water helps the self-perpetuation of continued generation of Free Radicals and their transformations after electrolysis. In other words, FRS water continues the electrolysis process itself for as long as the ORP levels are high. Therefore, FRS water can be used as a sterilizing solution for at least two- (2) hours after it is produced, which is a substantial practical benefit for use in comparison with the existing electrolysis water such as for example, Ultra Acidic Water. The ORP levels for Ultra Acidic Water is reduced immediately after production, which means a loss in sterilizing capability. Specifically, the Ultra Acidic Water can maintain a 900 mV ORP for only 10˜15 minutes after production. The ORP level of the FRS water of the present invention will reach its peak ORP usually 10˜15 minutes after production and maintain more than 900 mV level at least 2 hours thereafter, including the very high free radical reactions.

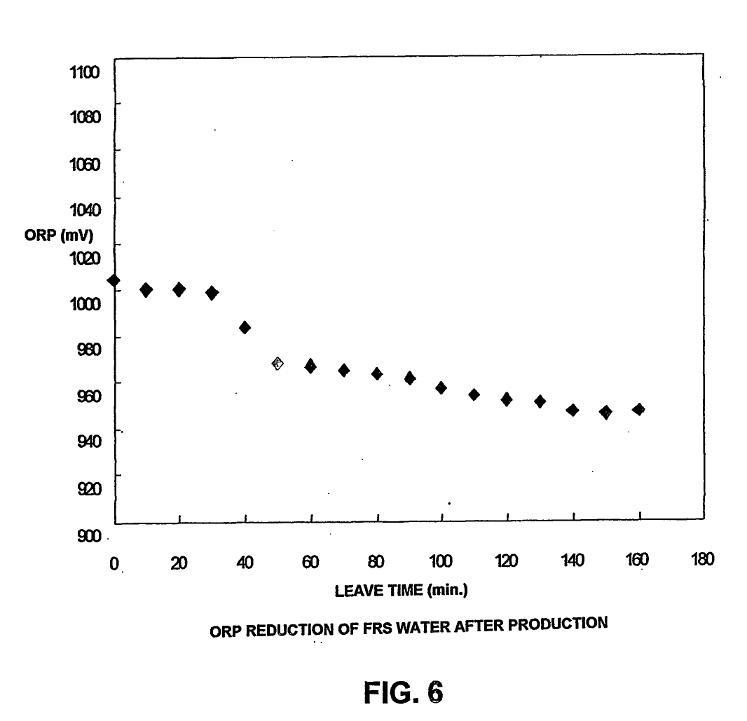

FIG. 6 is a graphical representation for the Oxidation-Reduction Potential change vs. time for FRS water. A peak of ORP is indicated at approximately 10 to 15 minutes after production, maintaining the same for about 40 minutes. The level of ORP, gradually decreases with time. However, very high level of ORP (at about 900 mV) is continuously maintained for more than 4 hours after production of FRS water. It should be noted that most microorganisms cannot live in environments with ORP levels of 900 mV or higher, and only a few can live between ORP levels of approximately 700-1000 mV. The FRS water maintains the high levels of ORP with a stabilized level of Hydrogen Ion Concentrate (HIC) of 6-8 pH. While FRS water gradually returns to regular water, Free Radicals continue to combine with other Free Radicals like oxygen found in FRS water and in the air. The exposure of FRS water to air increases the Dissolved Oxygen (DO) level, which facilitate to maintain the production of Free Radicals, due in part to increasing Oxidation Reduction Potential. In general, FRS water has more than 10 mg/L of DO. The production of Free Radicals eventually stop as the ORP levels in FRS water is decreased from about 1000 mV down to approximately 200 mV to 600 mV.

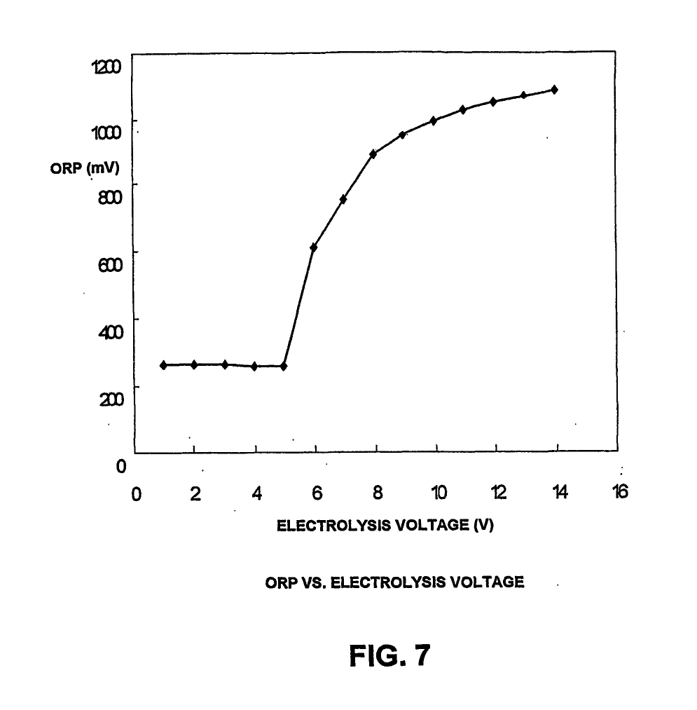

FIG. 7 is a graphical representation, illustrating power consumption during electrolysis for the production of FRS water. The Oxidation-Reduction Potential in FRS water increases as the electrolysis voltage is increased. This increase in ORP reduces the need for an ever-increasing application of power for continued electrolysis to generate FRS water, minimizing the power consumption during production. The amount of power applied to electrolyze water may be controlled either manually by a power supply and an amplifier, or automatically through a central processing unit. The relationship between the ORP level in FRS water and the application of power to produce the FRS water is dependent on many factors, including for example, the size of electrolysis cell, quality of Water, water temperature, etc. The Oxidation-Reduction Potential in FRS water may be adjusted by controlling the electric power to the electrolysis cell and/or the time-duration of electrolysis. For example, for use in medical environment, FRS water with highest ORP levels, such as 100 mV may be produced. For agriculture, 900 mV of ORP in FRS water may be sufficient. Since FRS water maintains ORP levels at more than 900 mV for at least two hours, the FRS water itself may be used as a source of electricity, for example, a battery.

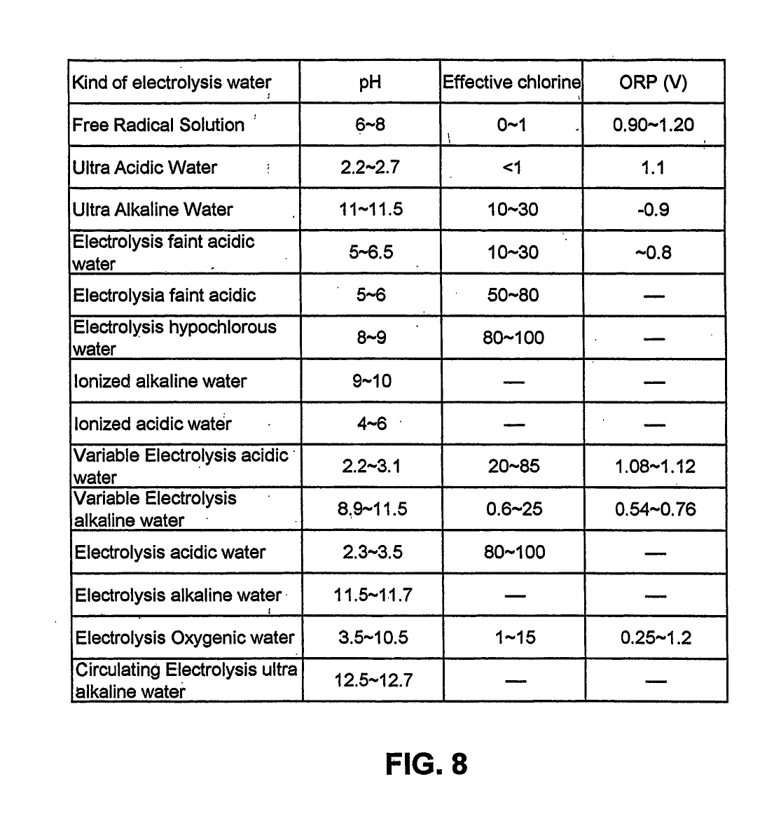

FIG. 8 is a comparison table illustrating the differences between the FRS water of the present invention and other existing prior art electrolysis water according to ORP, HIC, and Effective Chlorine levels. Prior art functional waters such as Ultra Acidic water use the chlorine contained in tap water to increase Oxidation Reduction Potential in the water solution. In addition, other additives including various chemicals are also added to improve the electrolytic character of water, which may cause the production of various chlorine gases harmful to human health. The FRS water production process of the present invention removes most of the chlorine at the first processing stage of water. Therefore, FRS water is generated as a result of transformation of water molecules, whereas all the other existing electrolysis waters are produced as a result of chemical reaction among water molecules and chemical electrolysis additives.

While illustrative embodiments of the invention have been described, numerous variations and alternative embodiments will occur to those skilled in the art. For example, small variations in Hydrogen Ion Concentration levels of 6-8 pH or the Oxidation-Reduction Potential of 900 mV to 1200 mV may be possible, due in part to the quality of water used to produce FRS water. Variations in Dissolved Oxygen levels will depend on many factors including temperature and atmospheric pressure, but it should be at least 10 mg/L. Such variations and alternate embodiments are contemplated, and can be made without departing from the spirit and the scope of the invention and the appended claims.

1. Functional water, comprising:
water molecules of H₂O, including free radicals;
stable Hydrogen Ion Concentration of 6 to 8 pH; and
Oxidation-Reduction Potential of 900 mV to 1200 mV.

2. Functional water according to claim 2, further comprising:
a service life time of at least two hours after production.

3. Functional water according to claim 1, wherein said functional water contains no additives during and after production.

4. Functional water according to claim 3, wherein said functional water is produced by pre-treatment and electrolysis of water.

5. Functional water according to claim 4, wherein said pre-treatment is comprised of purifying water prior to said electrolysis.

6. Functional water of claim 1, further comprising a Dissolved Oxygen level of greater than 10 mg/L.
